(19) United States
(12) Patent Application Publication
(10) Pub. No.: US 2009/0270844 A1
(43) Pub. Date: Oct. 29, 2009

(54) FLOW SENSOR CONTROLLED INFUSION DEVICE

(75) Inventors: Dale F. Seeley, Spring Park, MN (US); Timothy J. Denison, Minneapolis, MN (US)

Correspondence Address: CAMPBELL NELSON WHIPPS, LLC, 408 ST. PETER STREET, SUITE 240, ST. PAUL, MN, 55102, US

(73) Assignee: MEDTRONIC, INC., Minneapolis, MN (US)

(21) Appl. No.: 12/108,605

(22) Filed: Apr. 24, 2008

(51) Int. Cl. A61M 5/145

(52) U.S. Cl. 604/891.1

(57) ABSTRACT

An implantable infusion device includes an outlet through which a fluid is deliverable and a reservoir for containing the fluid. A flow path is in fluid communication with the reservoir and the outlet. The flow path includes a pressure regulator and a flow restrictor. The pressure regulator has a housing defining a major chamber and a diaphragm disposed in the housing such that the diaphragm sealingly divides the major chamber into first and second minor chambers. The flow restrictor is in fluid communication with the first and second minor chambers of the pressure regulator and is disposed downstream of the first minor chamber and upstream of the second minor chamber. The device further includes (i) a flow sensor configured to detect information regarding flow rate of the fluid downstream of the flow restrictor, and (ii) a pressure adjustment actuator assembly configured to vary pressure in the first minor chamber of the pressure regulator relative to pressure in the second minor chamber. The device also includes a processor operably coupled to the flow sensor and the pressure adjustment actuator assembly. The processor is configured to provide instructions to the actuator assembly for adjusting the pressure in the first minor chamber relative to pressure in the second minor chamber based on the information from the sensor to regulate flow rate of the fluid.

### FIELD

This disclosure relates to implantable medical devices, particularly implantable infusion devices employing flow sensors to regulate flow rate.

### BACKGROUND

A wide variety of implantable infusion devices are available for delivering fluid to target locations of a patient into which the device is implanted. Available and proposed devices can differ in their ability to control the flow rate of fluid delivered from the device to the patient. For example, Medtronic Inc.'s (Minneapolis, Minn.) SYNCHROMED series of implantable infusion devices are programmable devices where the flow rate may be varied according to instructions provided by, e.g., a physician programmer device. Medtronic Inc.'s SYNCHROMED implantable infusion devices employ peristaltic pumps that expel discrete amounts or spurts of fluid and can provide a wide range of fluid flow rates. Regardless of the pumping mechanism employed, fully programmable infusion devices are typically active devices requiring constant or near constant energy. The energy is typically supplied by a battery source, which increases the size of and cost to manufacture of the device. While fully programmable infusion devices allow for a wide variety of flow rates, they do at the expense of energy, size and design simplicity.

Other devices, such as Medtronic Inc.'s ISOMED implantable infusion device, are configured to deliver a relatively constant rate of fluid to the patient. Such constant rate devices are typically passive and are relatively simple in their components and construction. For example, the ISOMED device employs a fluid propellant source to force fluid out of a bellows reservoir and employs a capillary flow restrictor downstream of the positive pressure reservoir to control flow rate. The flow rate is dependent upon the fluidic resistance of the flow restrictor, temperature and viscosity of the fluid and the pressure differential across the restrictor, with pressure on one side being determined essentially by the pressure in the reservoir and pressure on the other side being determined essentially by body pressure, which closely follows atmospheric pressure. If any of fluidic resistance, temperature, viscosity or atmospheric pressure changes, the fluid flow rate can change. For example, changes in temperature or pressure associated with normal use of such devices can change flow rate 10-20%, which is unacceptable for a variety of therapies. Pressure regulator devices have been proposed to counteract the effect of ambient pressure change. However, the ability of such pressure regulators to counteract pressure change in a reliably consistent manner over time is in doubt. In addition, pressure regulators can require very precise/complex parts and fabrication to perform adequately.

Other infusion devices have been proposed that employ valves and a series of flow restrictors to convert a constant flow rate device into a selectable flow rate device. Such devices attempt to marry the simplicity of a constant rate device with the features of a fully programmable device. Some of the proposed selectable rate devices employ pressure sensors to determine the appropriate valves to open and close to direct fluid through a flow path with one or more flow restrictors to achieve a desired flow rate. However, such devices are still susceptible to changes in fluidic resistance, temperature, viscosity and pressure described above regarding constant rate infusion devices. That is, flow rate across any given flow restrictor may vary with, for example, a change in atmospheric pressure. Changes in pressure can result in feedback-controlled changes in flow path across a different flow restrictor, causing the device to make many active adjustments. Further, to account for differences in flow rate due to viscosity, temperature, and the like would require further components and design considerations in such devices, making their complexity more like programmable infusion devices.

In summary, programmable pumps often perform at a high level but typically require more energy, components, size and cost. Constant rate pumps are simple but do not work well in changing environments without adding complexity such as regulators. Selectable rate pumps loose some of the simplicity of the constant rate pumps and then become less differentiated from programmable pumps.

### BRIEF SUMMARY

The present disclosure describes, among other things, implantable infusion devices having a flow sensor feedback controlled fluid flow rate. The devices employ an actuator mechanism for controlling the relative pressure across a diaphragm of a pressure regulator or controlling a variable resistance valve to control flow rate. The devices may be used to provide a constant delivery rate or a variable delivery rate. In many embodiments, the complexity and manufacturing challenge of some of the components may be reduced due to the flow sensor feedback control.

In various embodiments, an implantable infusion device is described. The infusion device includes an outlet through which a fluid is deliverable and a reservoir for containing the fluid. A flow path is in fluid communication with the reservoir and the outlet. The flow path includes a pressure regulator and a flow restrictor. The pressure regulator has a housing defining a major chamber and a diaphragm disposed in the housing such that the diaphragm sealingly divides the major chamber into first and second minor chambers. The flow restrictor is in fluid communication with the first and second minor chambers of the pressure regulator and is disposed downstream of the first minor chamber and upstream of the second minor chamber. The device further includes (i) a flow sensor configured to detect information regarding flow rate of the fluid downstream of the flow restrictor, and (ii) an actuator assembly configured to vary pressure in the first minor chamber of the pressure regulator relative to pressure in the second minor chamber. The device also includes a processor operably coupled to the flow sensor and the actuator assembly. The processor is configured to provide instructions to the actuator assembly for adjusting the pressure in the first minor chamber relative to pressure in the second minor chamber based on the information from the sensor to regulate flow rate of the fluid. In some embodiments described herein, the pressure regulator is replaced by a variable valve configured to restrict fluid flow through the valve to varying degrees. The actuator assembly may be employed to adjust the degree to which the variable valve is opened.

In various embodiments, a method for controlling a flow rate of a fluid through a flow path of an implantable infusion device is described. The flow path includes a flow restrictor and a flow regulator. The flow regulator includes a major chamber sealingly divided into first and second minor chambers by a diaphragm. The flow restrictor is disposed in the flow path between the first and second minor chambers. The method includes sensing flow rate information downstream of the flow restrictor and determining whether the flow rate is at a target rate based on the sensed information. The method further includes adjusting the relative pressure between the first and second minor chambers of the pressure regulator if the flow rate is not at the target rate. If a variable valve is employed in place of a pressure regulator, the degree to which the valve is open may be adjusted.

Various embodiments of the present invention provide several advantages over known methods and apparatuses. For example, some of the embodiments described herein, provide for devices having fewer parts requiring tight tolerances than devices described previously. Such devices may, in some circumstances, be easier to manufacture than previously described implantable infusion devices. By providing a pressure adjustment actuator assembly coupled to a diaphragm of a flow regulator, a constant rate of delivery may be maintained over time, as the biasing force applied the diaphragm can be adjusted. Similarly, variable flow rate can be achieved with a high degree of accuracy by altering biasing force applied to the diaphragm. By employing a variable vale operably coupled to an adjustment actuator similar results may be obtained. These and other advantages will be evident to one of skill in the art upon reading the disclosure herein.

### BRIEF DESCRIPTION OF THE DRAWINGS

FIG. 1 is a schematic diagram showing an embodiment of an infusion device and operably coupled catheter implanted in a patient.

FIGS. 2-6 are schematic block diagrams of some components of implantable infusion devices showing representative flow paths and control components.

FIGS. 7A-B are schematic hybrids of block diagrams and cross-sectional views of an example of an actuator assembly and pressure regulator.

FIG. 8 is a flow diagram of a method for controlling a fluid flow rate of an implantable infusion device.

FIG. 9 is a flow diagram of a method for controlling a fluid flow rate of an implantable infusion device.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

The present disclosure describes, among other things, flow paths and control mechanisms for controllable rate infusion devices. The components described herein may be employed in a wide number of implantable infusion devices for delivering fluid to one or more target location of a patient in which the infusion device is implanted.

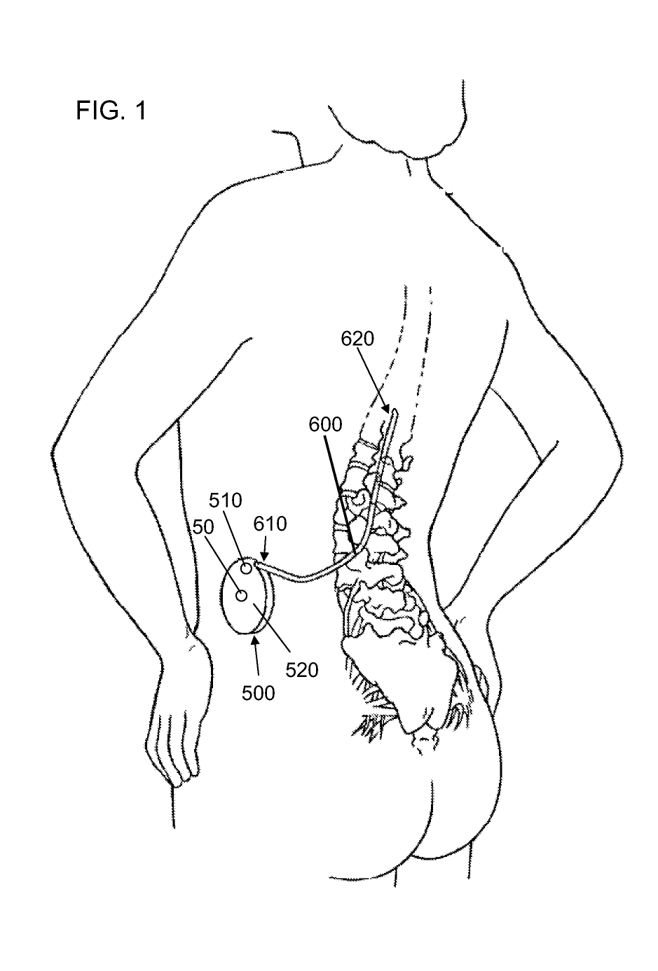

In various embodiments the implantable infusion device has a hermetically sealed housing in which some or all of the components are stored. For example and referring to FIG. 1, an implantable infusion device 500 and associated catheter 600 is shown implanted in a patient. In the depicted embodiment, the device 500 is implanted in a subcutaneous pocket in an abdominal region of the patient. However, it will be understood that the device may be implanted in any medically acceptable location of the patient. The device 500 includes a hermetically sealed housing 520 and a refill port 50 accessible from outside the housing 520. The refill port 50 provides access to the reservoir (not shown in FIG. 1). The reservoir may be refilled by percutaneously inserting a needle (not shown) into the patient such that needle enters refill port 50, and fluid may be delivered into reservoir from needle via refill port 50. The depicted device 500 also includes a catheter access port 510 in fluid communication with the catheter 600. Fluid may be injected into or withdrawn from the patient through catheter 600 via catheter access port 510 by percutaneously inserting a needle into access port 510. Catheter 600 is typically a flexible tube with a lumen running from the proximal end of catheter 610 to one or more delivery regions that are typically located at the distal portion of catheter 620. Proximal portion 610 of catheter 600 is connected to infusion device 500. Distal portion 620 of catheter 600 is positioned at a target location in the patient to deliver fluid from infusion device 500 to patient through a delivery region of catheter 600. While the system depicted in FIG. 1 is implanted to deliver fluid from the device 500 to the patient intrathecally via the catheter 600, it will be understood that the fluid can be delivered to any desired location.

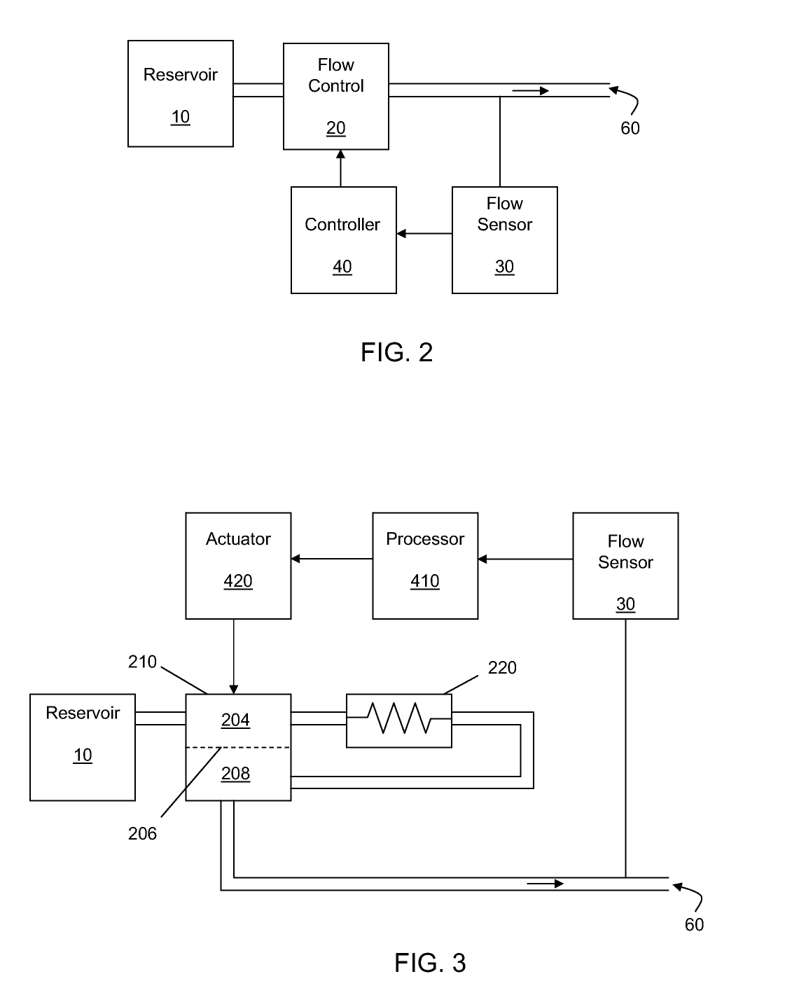

Referring now to FIG. 2, an overview of selected components of a controllable rate infusion device are shown in block diagram form. The device includes a reservoir 10 for storing fluid, which typically is a liquid composition including a therapeutic agent, and an outlet 60 through which the fluid can be delivered. The device includes a flow path extending from the reservoir 10 to the outlet 60. For purposes of the present disclosure, the reservoir 10 is discussed as being “upstream” of the outlet 60 in the flow path. The flow path includes a flow control mechanism 20 downstream of the reservoir 10 to control the flow rate of fluid delivered through the outlet 60. The device further includes a flow sensor 30 capable of detecting information regarding the flow rate of fluid that will exit opening 60. The flow sensor 30 may detect any information from which flow rate may be derived or estimated. For example, the flow sensor 30 may detect pressure upstream and downstream of a flow restrictor (not shown), may detect temperature upstream and downstream of a heating element (not shown), such as with a mass flow sensor, or the like. The device further includes a controller 40, such as processor or series of processors, which is capable of causing the flow control mechanism 20 to be adjusted based on information obtained from the sensor 30 to modify flow rate.

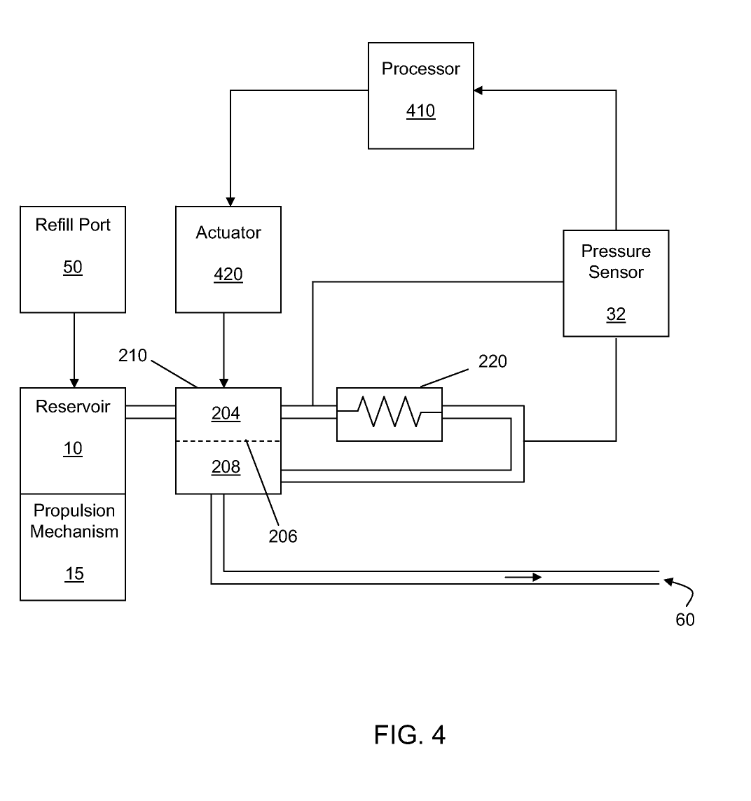

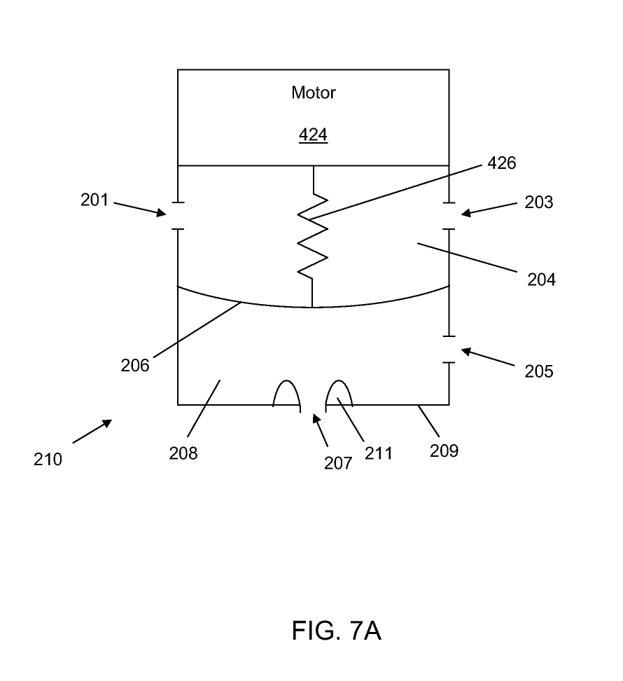

It will be readily apparent to those of ordinary skill in the art that a multitude of configurations of infusion devices including a reservoir, a flow control mechanism, a fluid flow path, a controller and a flow sensor may be employed to obtain a suitable controllable rate implantable infusion device. Examples of some configurations of such devices, or at least selected components of such devices, are shown in FIGS. 3-6. FIGS. 3-6 include additional details, relative to FIG. 2, of various embodiments of components of controllable rate infusion devices. In FIGS. 3-5, the flow path includes a pressure regulator 210 and flow restrictor 220. The pressure regulator 210 has a housing defining a major chamber and a diaphragm 206 disposed in the major chamber. The diaphragm 206 sealingly divides the major chamber into first 204 and second 208 minor chambers. The flow restrictor 220 is in fluid communication with the first 204 and second 208 minor chambers and is located in the flow path downstream of the first chamber 204 and upstream of the second chamber 208. The flow control mechanism includes an actuator assembly 420 configured to cause the diaphragm 206 to flex to control the relative pressure between the first chamber 204 and the second chamber 208 to control the rate of fluid flow through the flow path. Details regarding an embodiment of the interaction of the actuator assembly 420 and the pressure regulator 210 will be described below with regard to FIGS. 6A-B. The actuator assembly 420 in the embodiments depicted in FIGS. 3-5 receives instructions from processor 410 to change the position or bias on the diaphragm 206 to a greater or lesser degree. The processor 410 receives information from the flow sensor and instructs the actuator assembly 420 based on the information from the sensor.

It will be understood that regulator diaphragms without actuator adjustment are configured to change position based on pressure changes. However, with the actuator adjustment described herein, manufacturing of the pressure regulator and diaphragm may be less precise, allowing for the pressure regulator to provide gross adjustment with more precise adjustment provided by the actuator assembly based on flow sensor feedback. The configurations described herein also allow for improved performance over time, as the actuator assembly may compensate for degradation in material or system performance of the pressure regulator over time.

Any suitable flow restrictor 220 may be used in accordance with the embodiments depicted in FIGS. 3-6. For example, a flow restrictor may be a fluid conduit of restricted diametric dimension or a media to resist fluid flow. In some embodiments, the flow restrictor 220 is a capillary tube. In some embodiments, the flow restrictor 220 includes a fluidic path micro-machined in glass or silicon.

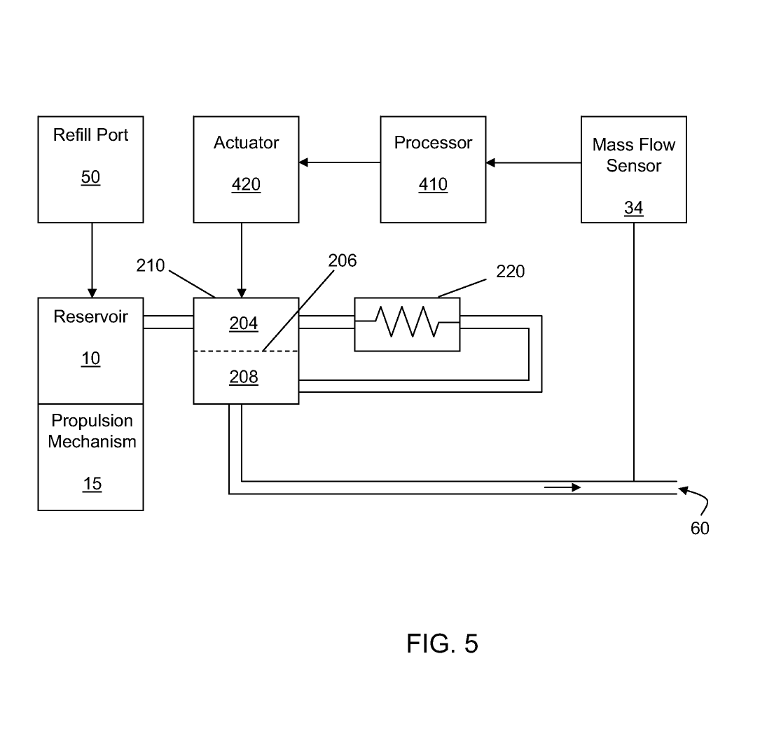

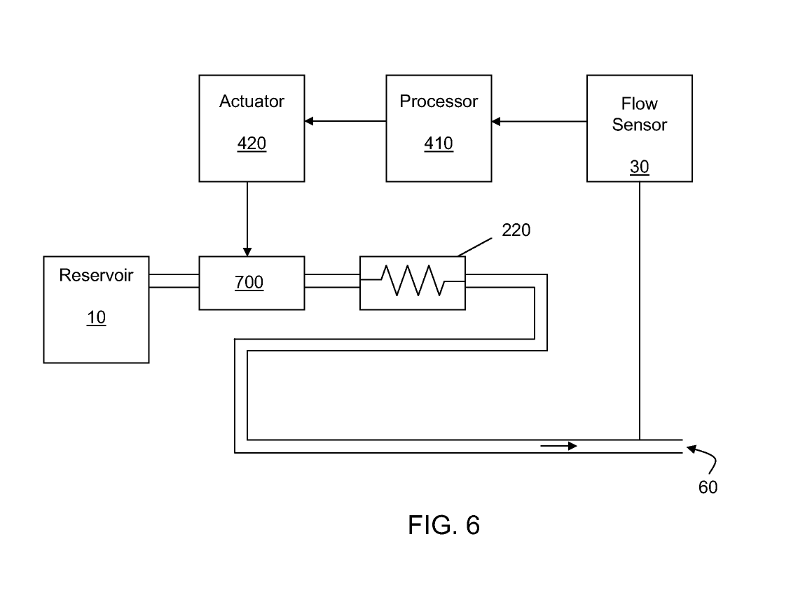

As depicted in FIGS. 4 and 5, a propulsion mechanism 15 may be operably coupled to the reservoir 10 to drive fluid out of the reservoir 10. Any suitable propulsion mechanism 15 may be employed. By way of example, the reservoir 10 may be a bellows reservoir and the propulsion mechanism 15 may contain a propellant chamber that contains a fluid whose vapor pressure is such that, under conditions of normal body temperature, pressure is exerted on the bellows to force liquid in the reservoir 10 to enter the pressure regulator 210. Examples of such propulsion mechanisms are found in Medtronic Inc.'s SYNCHROMED and ISOMED implantable infusion devices. A mechanical spring may be readily substituted for the liquid propellant. Alternatively, reservoir 10 may be formed, at least in part, of an elastomeric or resilient material biased in an empty configuration that expands when filled and forces fluid to exit reservoir 10 and enter regulator 210. Thus, the propulsion mechanism 15 and reservoir 10 may, in some embodiments, be the same component.

As shown in FIGS. 4 and 5, a refill port 50 may be included in an infusion device. The refill port 50 is in fluid communication with the reservoir 10 and provides access to the reservoir 10 to allow liquid to be delivered to, or withdrawn from, the reservoir 10. The refill port 50 may include a one-way valve mechanism (not shown) that allows fluid to be delivered to the reservoir 10 but prevents fluid from escaping the reservoir 10 via the access port 50. The refill port 50 may include check valve (not shown) or other mechanism to prevent overfilling of reservoir. One or more sensors (not shown) may be employed to detect needle entry into port 50, provide feedback regarding fill status of the reservoir 10, or the like.

As depicted in FIG. 4, flow sensor may include one or more pressure sensors 32. In the depicted embodiment, pressure sensor 32 senses pressure upstream and downstream of flow restrictor 220. Information regarding flow rate can be readily obtained by comparing pressure upstream and downstream of flow restrictor 220. Given a known viscosity of fluid flowing through the restrictor 220, the flow rate can readily be calculated by known algorithms based on the pressure differential across the restrictor 220. However, viscosity of fluid to be delivered to a patient from the reservoir 10 may depend on the composition of the fluid, internal body temperature of the patient into which the infusion device is implanted, or the like. Information regarding viscosity of the fluid composition may be provided to the infusion device by, e.g., an external device (not shown) in telemetric communication with the infusion device, for example when the reservoir 10 is refilled. The information may be stored in memory (not shown) and retrieved by processor 410 for use in determining whether or how much adjustment is appropriate and instructing actuator element 420 accordingly. A temperature sensor (not shown) may also be employed to provide information to processor 410, e.g. through memory or in real time, to account for a change in viscosity that may have occurred. While one pressure sensor 32 is depicted in FIG. 3, it will be understood that two pressure sensors may be employed, one upstream and one downstream of flow restrictor 220, both of which are operably coupled to processor 410. In some embodiments, pressure sensor 32 is a pressure transducer operably coupled to the flow path upstream and downstream of the restrictor 410. Of course, any suitable pressure sensor device may be employed.

As depicted in FIG. 5, flow sensor includes a mass flow sensor 34. Mass flow sensor 34 may be any suitable mass flow sensor. For example, the mass flow sensor 34 may include a heating or resistive element and two temperature sensors for measuring temperature of fluid upstream and downstream of the element. The sensor 34 (e.g. with on-board processor) or processor 410 may correlate the temperature difference to liquid mass flow. Such mass flow sensors are described in, for example, U.S. Pat. No. 6,813,944 to Mayer et al., issued on Nov. 9, 2004, assigned to Sensirion AG, and entitled “FLOW SENSOR”, which describes mass flow sensors that can detect liquid flow rates through a variety of materials, which patent is hereby incorporated herein by reference to the extent that it does not conflict with the present disclosure. Such sensors may be desirable to limit the number of components that come into contact with the liquid composition to be delivered to a patient. By limiting the number of components coming into contact with the liquid composition, concerns regarding the choice of material are reduced. For example, considerations such as whether the component may be affected by the liquid composition, whether the liquid composition may be affected by the component, and whether any safety concerns are presented in delivering a liquid that has contacted the component to a patient are reduced or eliminated if the component is not in contact with the liquid composition.

In some embodiments a pressure sensor device 32, e.g. as depicted in FIG. 4, is employed to provide information regarding fluid flow rate. In some embodiments a mass flow sensor device 34, e.g. as depicted in FIG. 5, is employed to provide information regarding fluid flow rate. As mass flow sensors provide a more direct measurement of flow rate that is not dependent on a correlation of pressure, temperature or fluidic resistance, infusion devices employing mass flow sensors may prove to be a bit more accurate.

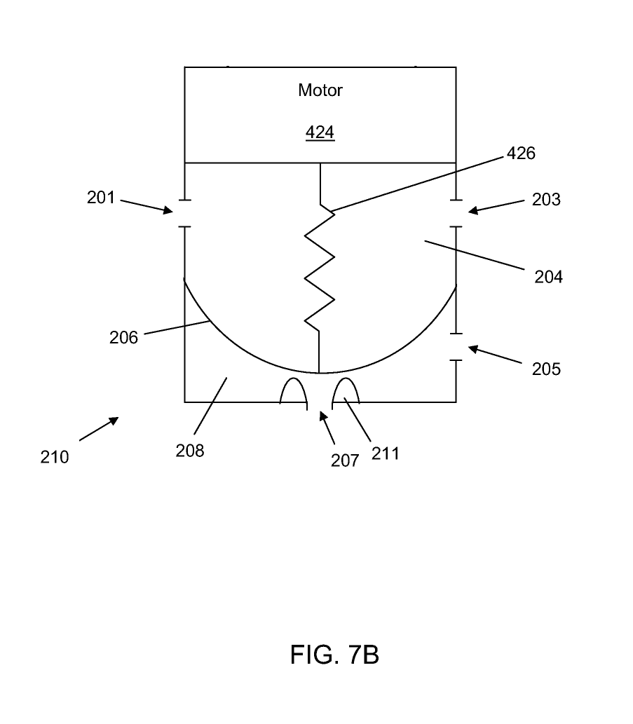

In various embodiments, a pressure regulator 210 as described above may be replaced with a variable valve. For example, and referring to FIG. 6, a variable valve 700 may be located upstream of a flow restrictor 220. Flow sensor 30 may detect information relating to flow rate and provide such information to processor 410, which in turn may instruct actuator 420 to adjust the amount that the valve is opened or closed. Any suitable actuatable valve assembly may be employed. For example, the valve may resemble a needle valve in a carburetor jet where a tapered needle is advanced into or withdrawn from a tapered opening to control flow. A ball valve or any other suitable valve may also be employed. The actuator mechanism 420 can be instructed to adjust the size of the opening of the variable valve 700 to change the rate at which fluid can flow through the valve 700. Otherwise, the components of the flow path, sensor 30, reservoir 10, processor 410 and actuator 420 depicted in FIG. 6 may be similar to those discussed above (e.g. with regard to FIGS. 2-5) or below (e.g. with regard to FIGS. 7A-B).

It will be understood that additional components that are not depicted in FIGS. 1-6 may be incorporated into an infusion device. For example, the device may include a power supply for powering the sensor 30, processor 410, actuator assembly 420 or the like. The device may include memory for storing information accessible by processor 410, storing information obtained from sensor 30, or the like. The device may include a telemetry module for communicating with a remote device, such as a programmer device, for example to obtain instructions regarding desired flow rate, viscosity of fluid stored in reservoir, or the like. It will be further understood that information obtained from flow sensor(s) 30 as described herein may also be used to provide diagnostic or other information, such as whether an occlusion exists along the flow path or how much liquid has been dispensed from the reservoir.

Referring now to FIGS. 7A-B, schematic hybrids of block diagrams and cross-sectional views of an example of an actuator assembly and pressure regulator are shown. In the depicted embodiment, the pressure regulator 210 includes a housing 209 defining a major chamber. A diaphragm 206 sealingly divides the major chamber into first 204 and second 208 minor chambers. An inlet 201 and an outlet 203 are in fluid communication with the first minor chamber 204. An inlet 205 and an outlet 207 are in fluid communication with the second minor chamber 208. As shown in FIGS. 3-5, a flow restrictor 220 may be disposed in the fluid flow path between the outlet 203 of the first minor chamber 204 and the inlet 205 of the second minor chamber 208. Diaphragm 206 is depicted in FIGS. 5A-B as a flexible element. However, it will be understood that the diaphragm may be supported by a bellows structure (not shown) to extend in a direction into the first chamber 204 or into the second chamber 208. In FIG. 5B, diaphragm 206 distends further into second chamber 208 relative to first chamber 204 than in FIG. 5B. Accordingly the pressure differential between the first chamber 204 and second chamber 208 is different. Specifically, the further diaphragm 206 distends into second chamber 208 the pressure fluid pressure in the second chamber 208 increases relative to the first chamber 204, thus affecting the pressure differential across the flow restrictor (see e.g. FIGS. 3-5) and affecting the flow rate of liquid through the flow path. If diaphragm 206 distends far enough into second chamber 208 diaphragm may engage sealing element 211, such as an o-ring, to cut off flow through outlet 207.

The diaphragm 206 may be formed of any suitable material that is impervious to the fluid delivered by the device. In an embodiment, the diaphragm 206 is formed of a thin foil metal such as titanium.

Referring now to the actuator assembly depicted in FIGS. 7A-B, assembly includes a motor 424 and an actuator element 426, such a spring, operably coupled to the motor 424. The actuator element 426 is also operably coupled to the diaphragm 206 and is capable of causing the diaphragm 206 to move, e.g. flex or expand, in a direction into the first chamber 204 or into the second chamber 208. The motor 424 may be a stepper motor, a shape memory alloy motor, or the like having a mechanism capable of translating rotational movement of the motor into linear compression or extension of the actuator element 426, such as a spring. As the actuator element 426 extends or contracts, the position of the diaphragm 206 changes to alter the relative pressure between the first chamber 204 and the second chamber 208. Such a control mechanism can provide for highly controlled and accurate dispensing of liquid from the infusion device. Such control mechanisms can be employed to ensure the accuracy of constant rate delivery of liquid from the device, if desired, or can be employed to adjust the rate of delivery as desired.

With regard to constant rate delivery, many of the propulsion mechanisms 15 described above with reference to FIGS. 4 and 5 are intended to supply a constant fluid flow rate but suffer from some drawbacks. For example, fluid rate delivery of liquid propellant based propulsion mechanisms can vary as ambient pressure varies. A patient may experience different flow rates from such devices depending on whether the patient is on an airplane, at 5000 feet above seal level, at sea level or below sea level. The use of the pressure adjustment actuator mechanism described herein allows the infusion device to compensate for changes in ambient conditions, such as ambient pressure, to ensure a more constant rate of delivery. By way of further example, spring force propulsion mechanisms tend to vary in the force applied around a force exhibited at a certain compression/extension state of the spring. That is, the force applied varies depending on the degree of extension or compression of the spring. In addition, the spring force applied by a spring may vary over time, generally becoming less with time. As such, the delivery rate of liquid from devices employing spring force propulsion mechanisms can vary. Propulsion mechanisms relying on resilient or elastic forces tend to have similar drawbacks as those employing spring force propulsion mechanisms. Regardless of the propulsion mechanism employed, the pressure adjustment actuator mechanisms described herein can be employed to improve consistency and accuracy of liquid dispensing from a constant rate delivery device.

It is worth noting that pressure regulators that do not employ a pressure adjustment actuator mechanism suffer from similar drawbacks to propulsion mechanisms relying on spring forces or resilient or elastic forces. As such, the devices described herein can result in implantable infusion devices with enhanced accuracy and controllability relative to prior devices or concepts.

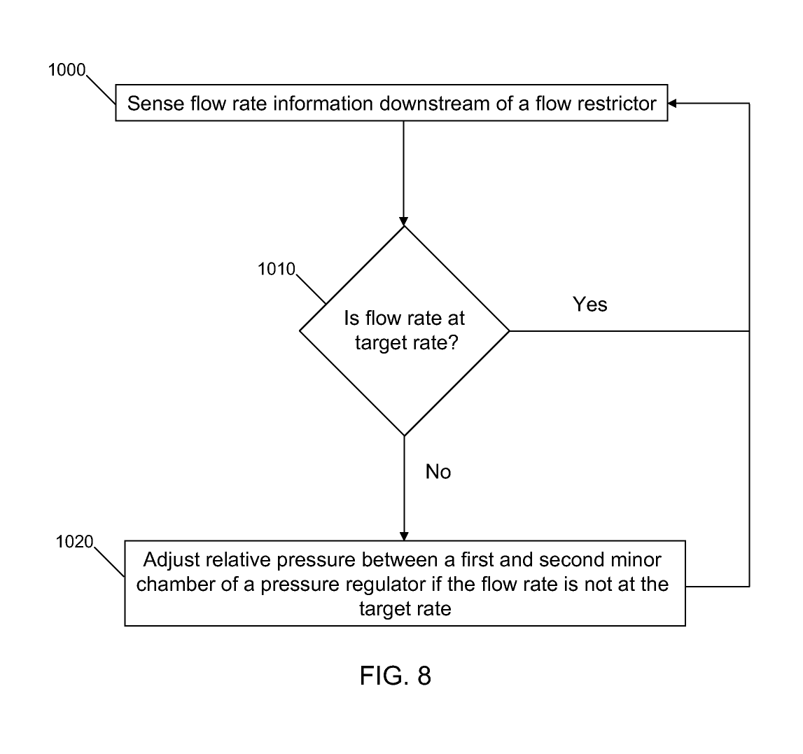

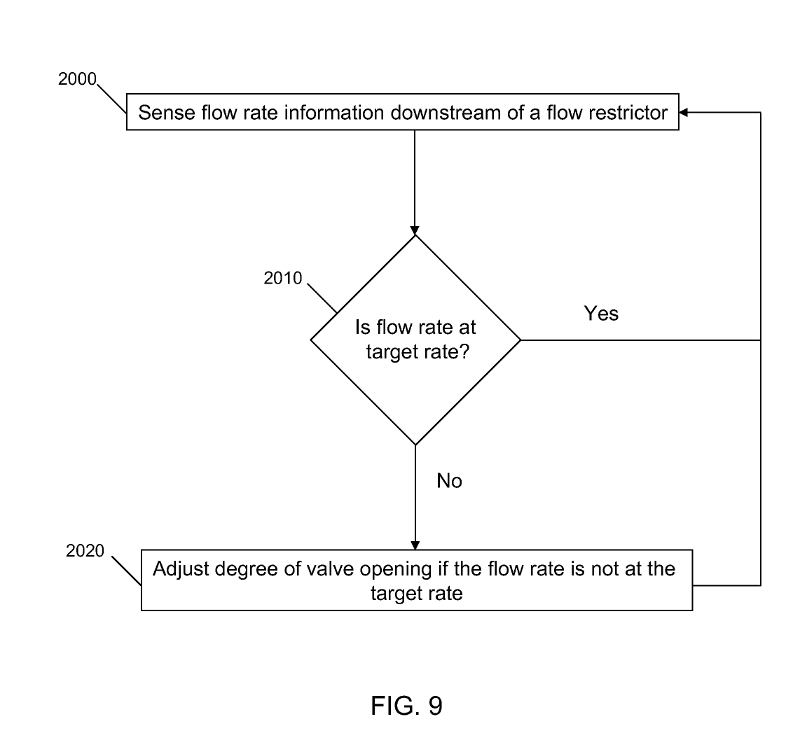

It will be understood that the components described in FIGS. 2-6 are but examples of components that an implantable infusion device may have and that many other device or system configurations may be employed to carry out the methods described below with regard to FIGS. 8-9. However, for the sake of convenience, the discussion that follows with regard to the methods illustrated in the flow diagram of FIGS. 8-9 will refer to components as described with regard to FIGS. 2-6.

Referring now to FIG. 8 an overview of a method for controlling flow rate is shown. In the depicted method, information regarding flow rate is sensed downstream of a flow restrictor 220 (1000). A determination is then made, based at least in part on the sensed information, as to whether the flow rate is at a target rate (1010). For example, processor 410 may compare information received from flow sensor 30 to information stored in memory regarding desired parameters to determine whether the flow rate is at the target rate. The method further includes adjusting the relative pressure between a first 204 and second 208 minor chamber of a flow regulator 210 if the flow rate is not at the target rate (1020). For example, processor 410 may instruct motor 424 to extend or contract actuator element 426 to adjust the degree to which diaphragm 206 is flexed to adjust the relative pressure between the minor chambers (204, 208). Information regarding the flow rate may continue to be monitored and further adjustments made as appropriate. The rate at which sampling and adjustment occur can be managed to conserve energy consumption.

Referring now to FIG. 9 an overview of a method for controlling flow rate employing a variable valve 700 is shown. In the depicted method, information regarding flow rate is sensed downstream of a flow restrictor 220 (2000). A determination is then made, based at least in part on the sensed information, as to whether the flow rate is at a target rate (2010). For example, processor 410 may compare information received from flow sensor 30 to information stored in memory regarding desired parameters to determine whether the flow rate is at the target rate. The method further includes adjusting the degree to which a variable valve 700 is open if the flow rate is not at the target rate (2020). For example, processor 410 may instruct a motor to extend or contract an actuator element to adjust the degree to which the variable valve 700 is open. Information regarding the flow rate may continue to be monitored and further adjustments made as appropriate.

Thus, embodiments of FLOW SENSOR CONTROLED INFUSION DEVICE are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. An implantable infusion device comprising:
an outlet through which a fluid is deliverable;
a reservoir for containing the fluid;
a flow path in fluid communication with the reservoir and the outlet, the flow path including:
(i) a pressure regulator having a housing defining a major chamber and a diaphragm disposed in the housing such that the diaphragm sealingly divides the major chamber into first and second minor chambers; and
(ii) a flow restrictor in fluid communication with the first and second minor chambers of the pressure regulator, wherein the flow restrictor is disposed downstream of the first minor chamber and upstream of the second minor chamber;
a flow sensor configured to detect information regarding flow rate of the fluid downstream of the flow restrictor;
an actuator assembly configured to vary pressure in the first minor chamber relative to pressure in the second minor chamber; and
a processor operably coupled to the flow sensor and the pressure adjustment actuator assembly, wherein the processor is configured to provide instructions to the actuator assembly for adjusting the pressure in the first minor chamber relative to pressure in the second minor chamber based on the information from the sensor to regulate flow rate of the fluid.

2. The device of claim 1, wherein the actuator assembly comprises an actuator element operably coupled to the diaphragm of the pressure regulator and is capable of causing the diaphragm to move to adjust the pressure in the first minor chamber relative to the second minor chamber.

3. The device of claim 2, wherein the actuator assembly further comprises a motor operably coupled to the actuator element and the processor, wherein the motor is capable of receiving the instructions from the processor for adjusting the pressure in the first minor chamber relative to pressure in the second minor chamber of the pressure regulator, and wherein the motor is capable of moving the actuator element to cause the diaphragm to move.

4. The device of claim 3, wherein the motor is capable of translating rotational movement of the motor or a component thereof into linear movement of the actuator element.

5. The device of claim 4, wherein the motor is a stepper motor.

6. The device of claim 4, wherein the motor is a shape memory motor.

7. The device of claim 2, wherein the actuator element comprises a spring.

8. The device of claim 1, wherein a sealing element is disposed within the second minor chamber of the pressure regulator, wherein the diaphragm, or a portion thereof, is capable of moving towards and engaging the sealing element to prevent fluid flow out of the second minor chamber.

9. The device of claim 1, wherein the reservoir or a portion thereof comprises a resilient material biased towards an empty position, wherein the reservoir or portion thereof distends when filled and forces fluid to exit the reservoir via the flow path.

10. The device of claim 1, wherein the reservoir is operably coupled to a propulsion mechanism to force fluid to exit the reservoir via the flow path.

11. The device of claim 10, wherein the propulsion mechanism comprises a propellant chamber containing a liquid having a vapor pressure at normal body temperature such that pressure is exerted on the reservoir to force fluid to exit the reservoir via the flow path.

12. The device of claim 1, wherein the flow sensor comprises a mass flow sensor.

13. A method for controlling flow rate of a fluid through a flow path of an implantable infusion device, the flow path including a flow restrictor and a flow regulator, the flow restrictor including a major chamber sealingly divided into first and second minor chambers by a diaphragm, wherein the flow restrictor is disposed in the flow path between the first and second minor chambers, the method comprising:
sensing flow rate information downstream of the flow restrictor;
determining whether the flow rate is at a target rate based on the sensed information; and
adjusting the relative pressure between the first and second minor chambers of the pressure regulator if the flow rate is not at the target rate.

14. The method of claim 13, wherein adjusting the relative pressure between the first and second minor chambers of the pressure regulator comprises moving the position of the diaphragm.

15. The method of claim 13, wherein moving the position of the diaphragm comprises moving an actuator element operably coupled to the diaphragm.

16. The method of claim 15, wherein moving the actuator element comprises translating rotational movement of a motor, or a component thereof, into linear movement of the actuator element.

17. The method of claim 13, wherein the method is carried out by the infusion device.

18. The method of claim 13, wherein the sensing the flow rate information downstream of the flow restrictor comprises sensing flow with a mass flow sensor.

19. An implantable infusion device comprising:
an outlet through which a fluid is deliverable;
a reservoir for containing the fluid;
a flow path in fluid communication with the reservoir and the outlet, the flow path including:
(i) a variable valve configure to allow variable amounts of fluid to pass through the valve; and
(ii) a flow restrictor in fluid communication with and disposed downstream of the variable valve;
a flow sensor configured to detect information regarding flow rate of the fluid downstream of the flow restrictor;
an actuator assembly configured to vary the degree to which the variable valve is open; and
a processor operably coupled to the flow sensor and the actuator assembly, wherein the processor is configured to provide instructions to the actuator assembly for adjusting the degree to which the variable valve is open based on the information from the sensor to regulate flow rate of the fluid.

20. The device of claim 19, wherein the flow sensor comprises a mass flow sensor.
